(19) United States
(12) Patent Application Publication
(10) Pub. No.: US 2025/0269146 A1
(43) Pub. Date: Aug. 28, 2025

(54) BI-DIRECTIONAL STEERABLE CATHETER

(71) Applicant: BOSTON SCIENTIFIC SCIMED, INC., MAPLE GROVE, MN (US)

(72) Inventors: Andrea Goering, Blaine, MN (US); David John Onushko, Maple Grove, MN (US); Christopher J Clark, St. Michael, MN (US); Sara Reda Eskander, Plymouth, MN (US); Matthew D. Himes, Ham Lake, MN (US)

(73) Assignee: BOSTON SCIENTIFIC SCIMED, INC., MAPLE GROVE, MN (US)

(21) Appl. No.: 19/062,740

(22) Filed: Feb. 25, 2025

Related U.S. Application Data
(60) Provisional application No. 63/557,744, filed on Feb. 26, 2024.

(51) Int. Cl. A61M 25/01; A61M 25/00

(52) U.S. Cl. CPC A61M 25/0147; A61M 25/0012; A61M 25/0045; A61M 2025/0008; A61M 25/0136; A61M 2207/00

(57) ABSTRACT

An elongate sheath may include an inner sleeve, a reinforcing braid over the inner sleeve, a pull ring between the inner sleeve and the braid, steering wires extending proximally from the pull ring, and an outer sleeve encompassing the braid. The steering wires extend between the inner sleeve and the braid to an exit location distal of the proximal ends of the inner and outer sleeves. A second reinforcing braid may be spaced apart from the braid proximal of the exit location. A method of making an elongate sheath may include positioning an inner sleeve on a mandrel, positioning a pull ring over the inner sleeve, wherein steering wires extend proximally from the pull ring, positioning a reinforcing braid over the inner sleeve, pull ring, and steering wires, positioning an outer sleeve over the braid, surrounding the outer sleeve with shrink wrap, and applying heat to reflow the outer sleeve.

### CROSS-REFERENCE TO RELATED APPLICATIONS

This application is a continuation of U.S. Patent Application Ser. No. 63/557,744, filed Feb. 26, 2024, entitled “BI-DIRECTIONAL STEERABLE CATHETER”, which is incorporated by reference herein in its entirety.

### TECHNICAL FIELD

The disclosure relates generally to medical devices and more particularly to mechanisms for steering catheters, sheaths, and/or elongate tubular shafts.

### BACKGROUND

A wide variety of intracorporeal medical devices have been developed for medical use, for example, surgical and/or intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods. There is an ongoing need to provide alternative medical devices as well as alternative methods for manufacturing and/or using medical devices.

### SUMMARY

In one example, an elongate sheath for a bi-directional steerable catheter may comprise an inner tubular sleeve having a proximal end and a distal end, a reinforcing braid disposed over the inner tubular sleeve between the proximal end of the inner tubular sleeve and the distal end of the inner tubular sleeve, a distal pull ring disposed radially between the inner tubular sleeve and the reinforcing braid, a first steering wire and a second steering wire each extending proximally from the distal pull ring, and an outer tubular sleeve encompassing the reinforcing braid, the outer tubular sleeve having a proximal end, a distal end, and a length defined by the proximal end and the distal end. The first steering wire and the second steering wire may extend between the inner tubular sleeve and the reinforcing braid from the distal pull ring proximally to an exit location disposed distal of the proximal end of the inner tubular sleeve. The proximal end of the outer tubular sleeve may be disposed proximal of the exit location.

In addition or alternatively to any example disclosed herein, a distal end of the reinforcing braid is spaced apart proximally from the distal end of the outer tubular sleeve and a proximal end of the reinforcing braid is disposed distal of the exit location.

In addition or alternatively to any example disclosed herein, the first steering wire and the second steering wire are fixedly attached to the distal pull ring.

In addition or alternatively to any example disclosed herein, the elongate sheath may comprise a first steering wire tube and a second steering wire tube each extending proximally from the distal pull ring to the exit location, the first steering wire and the second steering wire being slidably disposed within the first steering wire tube and the second steering wire tube, respectively.

In addition or alternatively to any example disclosed herein, the elongate sheath may comprise a distal marker band disposed distal of the distal end of the reinforcing braid and proximal of the distal end of the outer tubular sleeve.

In addition or alternatively to any example disclosed herein, the elongate sheath may comprise a proximal marker band spaced apart proximally from the distal marker band and spaced apart distally from the distal pull ring.

In addition or alternatively to any example disclosed herein, an elongate sheath for a bi-directional steerable catheter may comprise an inner tubular sleeve having a proximal end and a distal end, a reinforcing braid disposed over the inner tubular sleeve between the proximal end of the inner tubular sleeve and the distal end of the inner tubular sleeve, a distal pull ring disposed radially between the inner tubular sleeve and the reinforcing braid, a first steering wire and a second steering wire each extending proximally from the distal pull ring, and an outer tubular sleeve encompassing the reinforcing braid, the outer tubular sleeve having a proximal end, a distal end, and a length defined by the proximal end and the distal end. The first steering wire and the second steering wire may extend between the inner tubular sleeve and the reinforcing braid from the distal pull ring proximally to an exit location disposed distal of the proximal end of the inner tubular sleeve. The proximal end of the outer tubular sleeve may be disposed proximal of the exit location. The elongate sheath may comprise a second reinforcing braid spaced apart from the reinforcing braid. The second reinforcing braid may be disposed over the inner tubular sleeve proximal of the exit location and encompassed by the outer tubular sleeve.

In addition or alternatively to any example disclosed herein, the first steering wire and the second steering wire are disposed radially outward of the outer tubular sleeve proximal of the exit location.

In addition or alternatively to any example disclosed herein, in a normal configuration of the elongate sheath, a preset double curve is disposed between a proximal end of the reinforcing braid and a distal end of the reinforcing braid.

In addition or alternatively to any example disclosed herein, the outer tubular sleeve is formed from a plurality of individual segments of polymeric material.

In addition or alternatively to any example disclosed herein, at least three individual segments of the plurality of individual segments each have a different hardness and alternate longitudinally along the length of the outer tubular sleeve.

In addition or alternatively to any example disclosed herein, a method of making an elongate sheath for a bi-directional steerable catheter may comprise positioning an inner tubular sleeve on a mandrel; positioning a distal pull ring over the inner tubular sleeve proximal of a distal end of the inner tubular sleeve, wherein a first steering wire and a second steering wire each extend proximally from the distal pull ring; positioning a reinforcing braid over the inner tubular sleeve, the distal pull ring, the first steering wire, and the second steering wire; positioning an outer tubular sleeve over the reinforcing braid; surrounding the outer tubular sleeve with a shrink wrap; and applying heat to reflow the outer tubular sleeve around the reinforcing braid and the inner tubular sleeve.

In addition or alternatively to any example disclosed herein, positioning the reinforcing braid further comprises positioning a distal tubular braid tack over the reinforcing braid proximate a distal end of the reinforcing braid; positioning a distal shrink wrap over the distal tubular braid tack; and applying heat to reflow the distal tubular braid tack around the reinforcing braid proximate the distal end of the reinforcing braid.

In addition or alternatively to any example disclosed herein, positioning the reinforcing braid further comprises, after applying heat to reflow the distal tubular braid tack, positioning a proximal tubular braid tack over the reinforcing braid proximate a proximal end of the reinforcing braid; positioning a proximal shrink wrap over the proximal tubular braid tack; and applying heat to reflow the proximal tubular braid tack around the reinforcing braid proximate the proximal end of the reinforcing braid.

In addition or alternatively to any example disclosed herein, positioning the outer tubular sleeve further comprises positioning a plurality of individual segments of polymeric material over the reinforcing braid.

In addition or alternatively to any example disclosed herein, at least three individual segments of the plurality of individual segments each have a different hardness and alternate longitudinally along the length of the outer tubular sleeve.

In addition or alternatively to any example disclosed herein, positioning the outer tubular sleeve further comprises positioning a proximal handle segment of polymeric material over the inner tubular sleeve proximal of the reinforcing braid.

In addition or alternatively to any example disclosed herein, positioning the outer tubular sleeve further comprises positioning a second reinforcing braid over the inner tubular sleeve proximal of the reinforcing braid; and thereafter, positioning the proximal handle segment of polymeric material over the second reinforcing braid.

In addition or alternatively to any example disclosed herein, the proximal handle segment of polymeric material comprises a second reinforcing braid embedded therein.

In addition or alternatively to any example disclosed herein, the method may comprise fixedly attaching a hub member to a proximal end of the outer tubular sleeve.

In addition or alternatively to any example disclosed herein, the proximal handle segment is monolithically formed with a hub member disposed at a proximal end thereof.

In addition or alternatively to any example disclosed herein, a proximal portion of the first steering wire and a proximal portion of the second steering wire are disposed radially outward of the proximal handle segment of polymeric material.

The above summary of some embodiments, aspects, and/or examples is not intended to describe each embodiment or every implementation of the present disclosure. The figures and the detailed description more particularly exemplify aspects of these embodiments.

### BRIEF DESCRIPTION OF THE DRAWINGS

The disclosure may be more completely understood in consideration of the following detailed description in connection with the accompanying drawings, in which:

FIG. 1 illustrates selected aspects of an example steerable catheter;

FIG. 2 illustrates selected aspects of the example steerable catheter of FIG. 1;

FIG. 3 illustrates selected aspects of the example steerable catheter of FIG. 1;

FIG. 4 illustrates selected aspects of the example steerable catheter of FIG. 1;

FIG. 4A is a cross-sectional view of FIG. 4 taken along line 4A-4A;

FIG. 5 illustrates selected aspects of steering the example steerable catheter of FIG. 1 in a first direction;

FIG. 6 illustrates selected aspects of steering the example steerable catheter of FIG. 1 in a second direction;

FIGS. 7-12 illustrate selected aspects of an elongate sheath of the steerable catheter and a method of making the elongate sheath;

FIG. 13 illustrates selected aspects of an alternative configuration of the elongate sheath of the steerable catheter and a method of making the elongate sheath; and

FIG. 14 illustrates selected aspects of an alternative configuration of the elongate sheath of the steerable catheter and a method of making the elongate sheath.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified. The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

In some medical procedures, delivery and/or access sheaths may be routed percutaneously into a body cavity, lumen, and/or treatment site. Navigation through patient vasculature and/or organs may include steering through tortuous anatomy and/or directing a distal end of the delivery and/or access sheath into a body cavity, lumen, and/or treatment site. Examples of medical devices suitable for use in medical procedures, such as but not limited to left atrial appendage closure, aortic valve replacement, mitral valve replacement, septal defect repair, etc., are described herein. Existing medical devices may have certain advantages and/or disadvantages. There is an ongoing need for alternative steerable medical devices for delivering medical implants and/or conducting other treatment procedures.

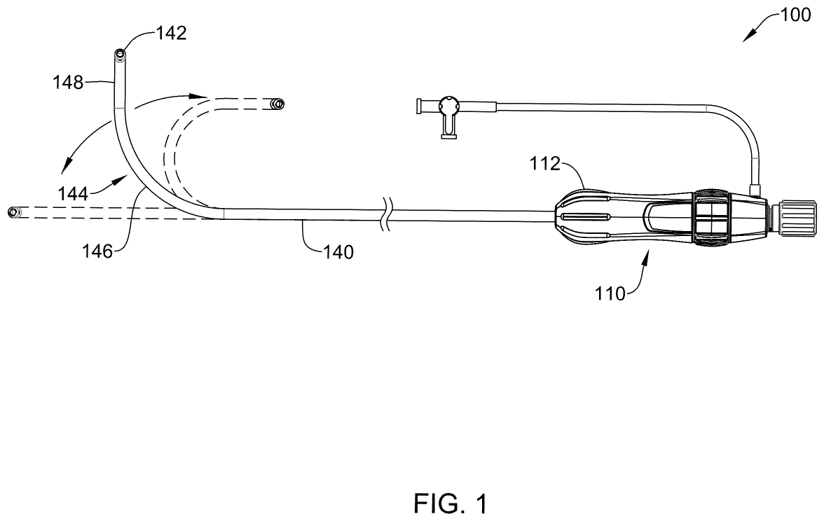

FIG. 1 illustrates selected aspects of a bi-directional steerable catheter 100. In some embodiments, the bi-directional steerable catheter 100 may be any one of a variety of catheters, such as an intravascular catheter. Examples of intravascular catheters may include, but are not limited to, balloon catheters, atherectomy catheters, device delivery catheters, drug delivery catheters, diagnostic catheters, and guide catheters. In some embodiments, the bi-directional steerable catheter 100 may take the form of other suitable guiding, diagnosing, or treating devices (including endoscopic instruments, laparoscopic instruments, etc., and the like) and it may be suitable for use at various locations and/or body lumens within a patient.

The bi-directional steerable catheter 100 may include a handle 110 and an elongate sheath 140 extending distally from the handle 110. In some embodiments, the bi-directional steerable catheter 100 and/or the handle 110 may include a guidewire port, a side port, a fluid flush port, an imaging access port, or other suitable ports, access points, or functional features. The handle 110 may include a handle housing 112. The elongate sheath 140 may extend into and/or through a distal opening in the handle housing 112. In at least some embodiments, a proximal end of the elongate sheath 140 may be fixedly attached to and/or may be disposed inside of the handle housing 112. In some embodiments, a proximal portion of the elongate sheath 140 may include a key element configured to non-rotatably engage one or more lock elements fixedly attached to an inner surface of the handle housing 112 proximal a distal end of the handle housing 112. In some embodiments, the key element may be bonded to an outer surface of the elongate sheath 140. In some embodiments, the key element may be integrally formed with the elongate sheath 140. In some embodiments, the key element may be welded (e.g., heat weld, sonic weld, vibration weld, etc.) to elongate sheath 140. In some embodiments, the key element may be melted together (e.g., reflowed) with the elongate sheath 140 such that material of the key element is co-mingled with material of the elongate sheath 140 at a molecular level. In some embodiments, the handle housing 112 may include one or more lock elements fixedly attached to and/or integrally formed with the inner surface of the handle housing 112. In some embodiments, the one or more lock elements may be formed as ribs or other structural support members configured to increase the rigidity of the handle housing and permit torque transfer between the distal end of the handle housing 112 and the elongate sheath 140. In some embodiments, the elongate sheath 140 may have a normal or relaxed configuration. The elongate sheath 140 may be self-biased toward, and/or in the absence of any outside forces may return to, the normal or relaxed configuration. Some suitable but non-limiting materials for the handle 110 and/or the handle housing 112 are described below.

In some embodiments, the elongate sheath 140 may include a soft and/or atraumatic distal tip 142. In some embodiments, the elongate sheath 140 may include a distal portion 144 having a first curve 146 and a second curve 148, such that the elongate sheath 140 has a preset double curve, in the normal or relaxed configuration, as shown in FIG. 1. In some embodiments, the first curve 146 may be preset to curve upwards, as viewed from the side. Other configurations are also contemplated. In some embodiments, the second curve 148 may be preset to curve to the left, as viewed proximally to distally along the elongate sheath 140. Other configurations are also contemplated. In some embodiments, the distal portion 144 and/or the first curve 146 may be configured to bend or deflect in a first direction, wherein the soft and/or atraumatic distal tip 142 is bent and/or moved towards and/or closer to the handle 110, toward and/or to a deflected configuration, as shown in phantom in FIG. 1. In some embodiments, the distal portion 144 and/or the first curve 146 may be configured to bend or deflect in a second direction opposite the first direction, wherein the soft and/or atraumatic distal tip 142 is bent and/or moved away from and/or farther from the handle 110, toward and/or to a straightened configuration, as shown in phantom in FIG. 1. In some embodiments, the elongate sheath 140 may have only a single curve in the normal or relaxed configuration. In some embodiments, the elongate sheath 140 may be substantially straight in the normal or relaxed configuration. Other configurations, including combinations of those described herein, are also contemplated.

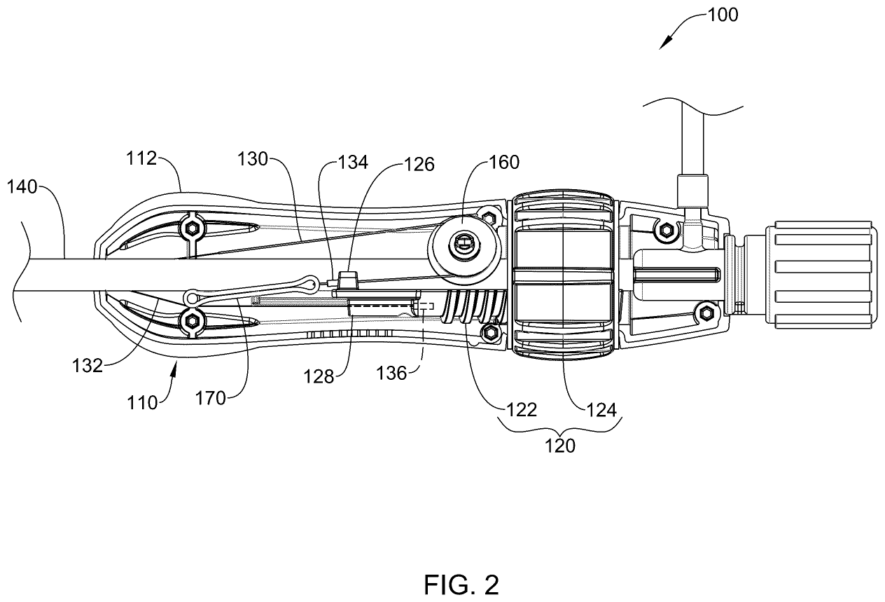

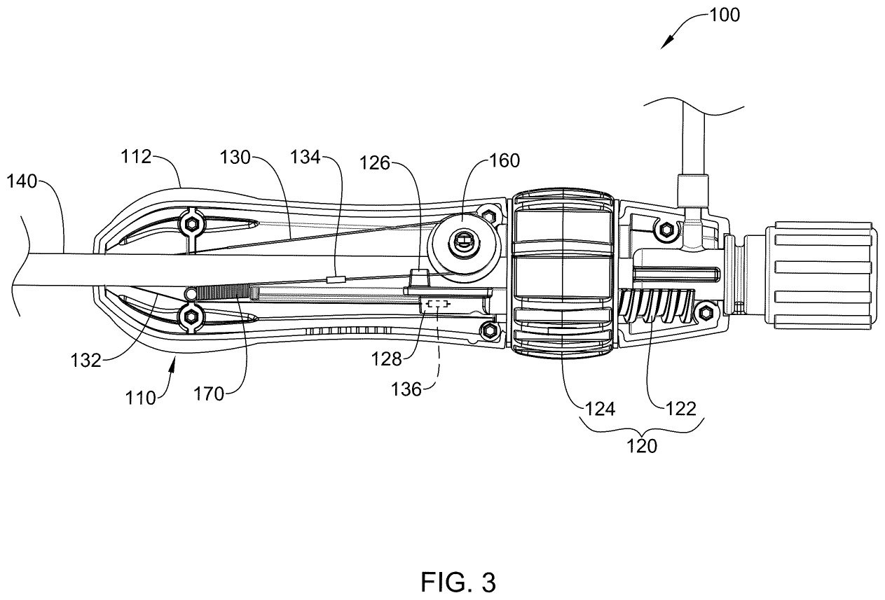

FIGS. 2 and 3 illustrate selected features of the bi-directional steerable catheter 100. In the view shown, a portion of the handle housing 112 has been removed to show some internal components of the handle 110. In some embodiments, the handle 110 may include an axial translation mechanism 120. In some embodiments, the axial translation mechanism 120 may include a threaded member 122 slidably disposed within the handle 110 and/or the handle housing 112. In some embodiments, axial translation mechanism 120 may include a rotatable knob 124. In some embodiments, the rotatable knob 124 may be disposed about and/or may be configured to rotate about, and/or relative to, at least a portion of the handle 110 and/or the handle housing 112. In some embodiments, the rotatable knob 124 may be configured to engage the threaded member 122 such that rotation of the rotatable knob 124 relative to the handle 110 and/or the handle housing 112 causes axial translation of the threaded member 122 proximally and/or distally within the handle 110 and/or the handle housing 112. In some embodiments, rotation of the rotatable knob 124 in a clockwise direction, as viewed along the bi-directional steerable catheter 100 proximally to distally, may cause axial translation of the threaded member 122 distally within the handle 110 and/or the handle housing 112. In some embodiments, rotation of the rotatable knob 124 in a counterclockwise direction, as viewed along the bi-directional steerable catheter 100 proximally to distally, may cause axial translation of the threaded member 122 proximally within the handle 110 and/or the handle housing 112. In some embodiments, the reverse and/or opposite configuration may be used, wherein clockwise rotation of the rotatable knob 124 moves the threaded member 122 proximally and counterclockwise rotation of the rotatable knob 124 moves the threaded member 122 distally. The orientation of the internal and external threads on the rotatable knob 124 and the threaded member 122, respectively, determine which direction of rotation is tied to which direction of axial translation. Some suitable but non-limiting materials for the axial translation mechanism 120, the threaded member 122, and/or the rotatable knob 124 are described below.

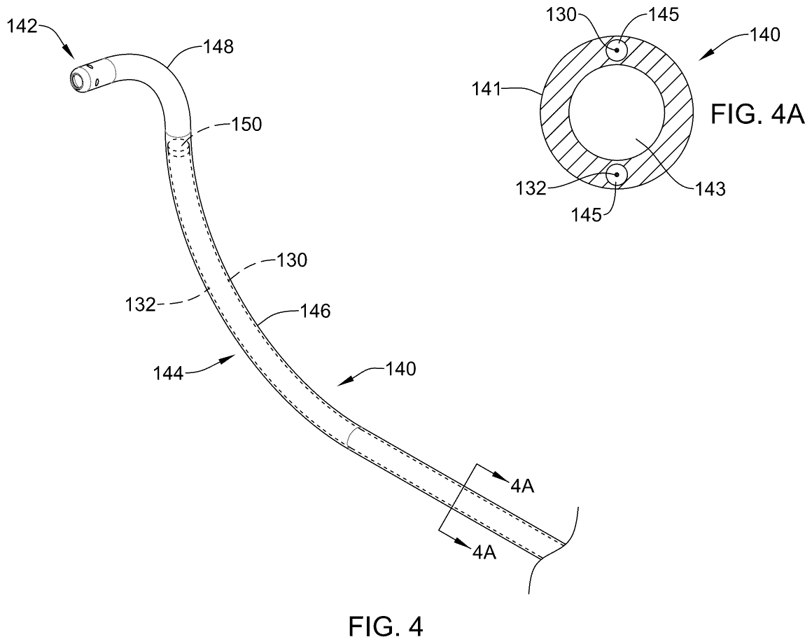

A first steering wire 130 may extend through the elongate sheath 140 from the handle 110 and/or the handle housing 112 to a distal pull ring 150 (e.g., FIG. 4). A second steering wire 132 may extend through the elongate sheath 140 from the handle 110 and/or the handle housing 112 to the distal pull ring 150 (e.g., FIG. 4). The second steering wire 132 may be disposed on an opposite side of the elongate sheath 140 from the first steering wire 130 relative to a central longitudinal axis of the elongate sheath 140. Tension may be applied to the first steering wire 130 and/or the second steering wire 132 as described herein to bend and/or deflect the distal portion 144 and/or the first curve 146 of the elongate sheath 140 (e.g., FIG. 1). The first steering wire 130 may be configured to engage the axial translation mechanism 120 and/or the threaded member 122 to bend and/or deflect the distal portion 144 and/or the first curve 146 of the elongate sheath 140 in the first direction toward the handle 110 and/or the handle housing 112, toward and/or to the deflected configuration (e.g., FIG. 1). The second steering wire 132 may be configured to engage the axial translation mechanism 120 and/or the threaded member 122 to bend and/or deflect the distal portion 144 and/or the first curve 146 of the elongate sheath 140 in the second direction opposite the first direction and away from the handle 110 and/or the handle housing 112, toward and/or to the straightened configuration (e.g., FIG. 1).

In some embodiments, the bi-directional steerable catheter 100 may include a pulley wheel 160 disposed within the handle 110 and/or the handle housing 112. The pulley wheel 160 may be engaged with the first steering wire 130 via a circumferential channel extending around the pulley wheel 160. In some embodiments, the pulley wheel 160 may engage the first steering wire 130 at a position proximate a distal end of the threaded member 122. In some embodiments, the pulley wheel 160 may engage the first steering wire 130 at a position proximal of a distal end of the threaded member 122. In some embodiments, the bi-directional steerable catheter 100 may include a tensioning member 170. The tensioning member 170 may couple a first end (e.g., a proximal end) of the first steering wire 130 to the handle 110 and/or to the handle housing 112. In at least some embodiments, the proximal end of the first steering wire 130 may be fixedly coupled to the handle 110 and/or the handle housing 112 by the tensioning member 170. In some embodiments, the pulley wheel 160 may engage the first steering wire 130 at a position proximal of the tensioning member 170. In some embodiments, the tensioning member 170 may be coupled to the handle 110 and/or the handle housing 112 at a position distal of the proximal end of the first steering wire 130. In some embodiments, the tensioning member 170 may be an elastic polymer, as shown in FIG. 2. In another example, the tensioning member 170 may be a coil spring, as shown in FIG. 3. Other configurations are also contemplated. As will be apparent, the tensioning member 170 may be configured to apply a small, non-biasing amount of tension to the first steering wire 130 when the distal portion 144 and/or the first curve 146 of the elongate sheath 140 is disposed in the normal or relaxed configuration and/or when the distal portion 144 and/or the first curve 146 of the elongate sheath 140 is bent and/or deflected in the second direction, toward and/or to the straightened configuration. The purpose of the tensioning member 170 is to prevent the first steering wire 130 from disengaging from the pulley wheel 160 when there is no tension being applied to the first steering wire 130 by the axial translation mechanism 120 and/or the threaded member 122 (e.g., in the normal or relaxed configuration, or toward and/or in the straightened configuration) by holding the first steering wire 130 taught around the pulley wheel 160. Some suitable but non-limiting materials for the pulley wheel 160 and/or the tensioning member 170 are described below.

In addition or alternatively, in some embodiments, the bi-directional steerable catheter 100 may include one or more ribs, projections, bosses, or posts extending transversely within the handle housing 112 between opposing walls and/or opposite sides of the handle housing 112. In some embodiments, the one or more ribs, projections, bosses, or posts may be disposed within the handle housing 112 at positions configured to approximate the diameter and/or the perimeter of the pulley wheel 160. In some embodiments, the one or more ribs, projections, bosses, or posts may replace the pulley wheel 160. In some embodiments, the one or more ribs, projections, bosses, or posts may be provided in addition to the pulley wheel 160. In some embodiments, the one or more ribs, projections, bosses, or posts may extend completely across an interior of the handle housing 112 from one side of the handle housing 112 to an opposing side of the handle housing 112. In some embodiments, the first steering wire 130 may be routed around and/or may slide past the one or more ribs, projections, bosses, or posts in a manner similar to the first steering wire 130 extending around the pulley wheel 160, such that the one or more ribs, projections, bosses, or posts may serve as guides for the first steering wire 130 and prevent loss of motion.

The threaded member 122 may include a first catch 126 extending transversely from the threaded member 122 in a first lateral direction. The first steering wire 130 may extend and/or pass through the first catch 126. The first steering wire 130 may include a first stop element 134 configured to engage with the axial translation mechanism 120 and/or the first catch 126 of the threaded member 122 when the threaded member 122 slides in a distal direction within the handle 110 and/or the handle housing 112 to apply tension to the first steering wire 130, as seen in FIG. 2. The tension applied by the axial translation mechanism 120 and/or the threaded member 122 may be sufficient to overcome the self-bias of the elongate sheath 140 toward the normal or relaxed configuration and bend and/or deflect the distal portion 144 and/or the first curve 146 of the elongate sheath 140 in the first direction.

The threaded member 122 may include a second catch 128 extending transversely from the threaded member 122 in a second lateral direction opposite the first lateral direction. The second steering wire 132 may extend and/or pass through the second catch 128. The second steering wire 132 may include a second stop element 136 configured to engage with the axial translation mechanism 120 and/or the second catch 128 of the threaded member 122 when the threaded member 122 slides in a proximal direction within the handle 110 and/or the handle housing 112 to apply tension to the second steering wire 132, as seen in FIG. 3. The tension applied by the axial translation mechanism 120 and/or the threaded member 122 may be sufficient to overcome the self-bias of the elongate sheath 140 toward the normal or relaxed configuration and bend and/or deflect the distal portion 144 and/or the first curve 146 of the elongate sheath 140 in the second direction.

The pulley wheel 160 permits the threaded member 122 to apply tension to both the first steering wire 130 and the second steering wire 132, depending upon which direction the threaded member 122 is moving. Tension applied to the first steering wire 130 and the second steering wire 132 causes bending and/or deflection of the distal portion 144 and/or the first curve 146 of the elongate sheath 140 away from the normal or relaxed configuration. Since both steering wires extend proximally from the distal pull ring 150, the pulley wheel 160 is needed to reverse the direction of the first steering wire 130 relative to the second steering wire 132 within the handle 110 and/or the handle housing 112 such that the threaded member 122 is able to selectively apply tension to both the first steering wire 130 and the second steering wire 132 by moving in opposite directions. In one or more alternative configurations, the handle 110 and/or the handle housing 112 may include an internal rib, an internal protrusion, or other features disposed therein, in place of the pulley wheel 160, around which the first steering wire 130 may extend and reverse direction to function as described herein.

When the threaded member 122 is disposed in a central position, the distal portion 144 and/or the first curve 146 of the elongate sheath 140 may be disposed in the normal or relaxed configuration. When the threaded member 122 is disposed in the central position, substantially no tension is being applied to the first steering wire 130 and/or the second steering wire 132. As the threaded member 122 is axially translated proximally and/or distally within the handle 110 and/or the handle housing 112, the threaded member 122 of the axial translation mechanism 120 may engage with the first steering wire 130 and/or the second steering wire 132 to apply tension thereto to bend and/or deflect the distal portion 144 and/or the first curve 146 of the elongate sheath 140 as described herein. Additionally, when the threaded member 122 is disposed in the central position, the first catch 126 may be engaged with the first stop element 134 but tension is not being applied to the first steering wire 130, and the second catch 128 may be engaged with the second stop element 136 but tension is not being applied to the second steering wire 132. As such, the central position of the threaded member 122 may be tension-neutral with respect to the first steering wire 130 and the second steering wire 132.

When the threaded member 122 is moved from the central position toward and/or until disposed in a proximal position, tension may be applied to the second steering wire 132 and the distal portion 144 and/or the first curve 146 of the elongate sheath 140 may be bent and/or deflected in the second direction away from the handle 110 and/or the handle housing 112, or toward and/or to the straightened configuration. In moving the threaded member 122 proximally within the handle 110 and/or the handle housing 112 from the central position, the second catch 128 engages the second stop element 136 and thereafter translates the second stop element 136 proximally, thereby applying tension to the second steering wire 132, as seen in FIG. 3. The first stop element 134 may disengage from the axial translation mechanism 120, the threaded member 122, and/or the first catch 126 to release tension on the first steering wire 130 when the threaded member 122 slides in the proximal direction within the handle 110 and/or the handle housing 112. Accordingly, when the threaded member 122 is moved proximally from the central position, the first catch 126 may be disengaged from the first stop element 134 and the first catch 126 may slide proximally along and/or over the first steering wire 130. The first stop element 134 may be configured to float relative to (e.g., the first stop element 134 may not be directly fixed to) the axial translation mechanism 120, the threaded member 122, and/or the first catch 126 when the threaded member 122 slides in the proximal direction within the handle 110 and/or the handle housing 112. As such, slack would form in the first steering wire 130, which would allow the first steering wire 130 to disengage from the pulley wheel 160, except for the tension applied by the tensioning member 170. The tensioning member 170 holds the first steering wire 130 tightly around the pulley wheel 160 while no tension is being applied to the first steering wire 130 by the threaded member 122 and/or the first catch 126. The tensioning member 170 merely absorbs any slack that would be formed in the first steering wire 130 due to the first catch 126 being disengaged from the first stop element 134 and prevents the first steering wire 130 from disengaging from the pulley wheel 160. This feature may be seen in the configuration shown in FIG. 3, for example.

When the threaded member 122 is moved from the central position toward and/or until disposed in a distal position, tension may be applied to the first steering wire 130 and the distal portion 144 and/or the first curve 146 of the elongate sheath 140 may be bent and/or deflected in the first direction toward the handle 110 and/or the handle housing 112, or toward and/or to the deflected configuration. In moving the threaded member 122 distally within the handle 110 and/or the handle housing 112 from the central position, the first catch 126 engages the first stop element 134 and thereafter translates the first stop element 134 distally, thereby applying tension to the first steering wire 130, as seen in FIG. 2. The second stop element 136 may disengage from the axial translation mechanism 120, the threaded member 122, and/or the second catch 128 to release tension on the second steering wire 132 when the threaded member 122 slides in the distal direction within the handle 110 and/or the handle housing 112. Accordingly, when the threaded member 122 is moved distally from the central position, the second catch 128 may be disengaged from the second stop element 136 and the second catch 128 may slide distally along and/or over the second steering wire 132. The second stop element 136 may be configured to float relative to (e.g., the second stop element 136 may not be directly fixed to) the axial translation mechanism 120, the threaded member 122, and/or the second catch 128 when the threaded member 122 slides in the distal direction within the handle 110 and/or the handle housing 112. As such, slack forms in the second steering wire 132, as may be seen in the configuration shown in FIG. 2, due to the second catch 128 being disengaged from the second stop element 136. As the threaded member 122 is translated distally from the proximal position and/or the central position, the first catch 126 engages the first stop element 134 and the first steering wire 130 is thereafter pulled around the pulley wheel 160 and tension applied by the tensioning member 170 is relieved as tension is instead applied to the first steering wire 130 by the first catch 126 and/or the threaded member 122.

FIGS. 4 and 4A illustrate selected aspects of an example configuration for the elongate sheath 140, as least some of which are described above. In some embodiments, the elongate sheath 140 may include a wall 141 defining a central lumen 143 extending from a proximal end to the soft and/or atraumatic distal tip 142 along the central longitudinal axis of the elongate sheath 140. In at least some embodiments, the central lumen 143 may be coaxial with the central longitudinal axis of the elongate sheath 140. In some embodiments, the central lumen 143 may be a guidewire lumen. In some embodiments, the central lumen 143 may be a device lumen used to deliver a medical device or implant. In some embodiments, the central lumen 143 may have multiple uses. The elongate sheath 140 may include a plurality of steering wire lumens 145 extending and/or disposed within the wall 141. In some embodiments, the plurality of steering wire lumens 145 may include a first steering wire lumen and a second steering wire lumen. In some embodiments, the plurality of steering wire lumens 145 may include more than two steering wire lumens. In some embodiments, the plurality of steering wire lumens 145 may be oriented substantially parallel to the central lumen 143 and/or the central longitudinal axis of the elongate sheath 140. In some embodiments, the plurality of steering wire lumens 145 may be disposed opposite each other and/or on opposite sides of the elongate sheath 140 relative to the central lumen 143 and/or the central longitudinal axis of the elongate sheath 140. Other configurations are also contemplated.

In some embodiments, the plurality of steering wire lumens 145 may have a circular cross-sectional shape, as shown in FIG. 4. However, the cross-sectional shape shown in FIG. 4 is merely exemplary and is not intended to be limiting. In some embodiments, the plurality of steering wire lumens 145 may have other cross-sectional shapes. For example, in some embodiments, the plurality of steering wire lumens 145 may have a rectangular cross-sectional shape, an ovoid cross-sectional shape, a square cross-sectional shape, a polygonal cross-sectional shape, etc. In some embodiments, the plurality of steering wire lumens 145 may have a cross-sectional shape that is regular and/or symmetrical. In some embodiments, the plurality of steering wire lumens 145 may have a cross-sectional shape that is irregular and/or asymmetrical. Other configurations are also contemplated.

As discussed herein, a distal pull ring 150 may be disposed within the distal portion 144 of the elongate sheath 140. In some embodiments, the distal pull ring 150 may be disposed proximal to the second curve 148 and/or the soft and/or atraumatic distal tip 142. In at least some embodiments, the distal pull ring 150 may be disposed proximate a distal end of the first curve 146. In some embodiments, the distal pull ring 150 may be embedded within the wall 141 of the elongate sheath 140. In some embodiments, the distal pull ring 150 may be secured, bonded, and/or fixedly attached to an inner surface of the wall 141 of the elongate sheath 140. Other configurations are also contemplated. Some suitable but non-limiting materials for the distal pull ring 150 are described below.

The first steering wire 130 and the second steering wire 132 may each be slidably disposed within the plurality of steering wire lumens 145. In one example, the first steering wire 130 may be slidably disposed within the first steering wire lumen and the second steering wire 132 may be disposed within the second steering wire lumen. The first steering wire 130 and the second steering wire 132 may be fixedly attached (e.g., bonded, welded, etc.) to the distal pull ring 150. For example, a distal end of the first steering wire 130 may be fixedly attached to the distal pull ring 150 and a distal end of the second steering wire 132 may be fixedly attached to the distal pull ring 150 at a position opposite the distal end of the first steering wire 130 relative to the central longitudinal axis of the elongate sheath 140. Some suitable but non-limiting materials for the first steering wire 130 and the second steering wire 132 are described below.

In some embodiments, the elongate sheath 140 may be sized in accordance with its intended use. For example, the elongate sheath 140 can have a length that is in the range of about 50 to about 200 centimeters, about 75 to about 175 centimeters, or about 100 to about 150 centimeters. Other lengths are also contemplated. It is further contemplated that the outer diameter of the elongate sheath 140 may vary based on the use or application. In some examples, the outer diameter of the elongate sheath 140 may be about 2 millimeters (mm), about 3 mm (or 9 French), about 3.5 mm, about 4 mm (or 12 French), about 4.5 mm, about 5 mm (or 15 French), about 5.33 mm, about 5.5 mm, about 5.66 mm (or 17 French), about 6 mm, about 6.5 mm, about 7 mm (or 21 French), about 8 mm, or other suitable sizes. In some embodiments, the outer diameter of the elongate sheath 140 may be a maximum of 5.66 mm (17 French), and is preferably smaller than 5.66 mm (17 French). Other configurations are also contemplated. Additional aspects, constructional details, and/or methods or steps related to making and/or manufacturing the elongate sheath 140 are discussed below. Some suitable but non-limiting materials for the elongate sheath 140 are described below.

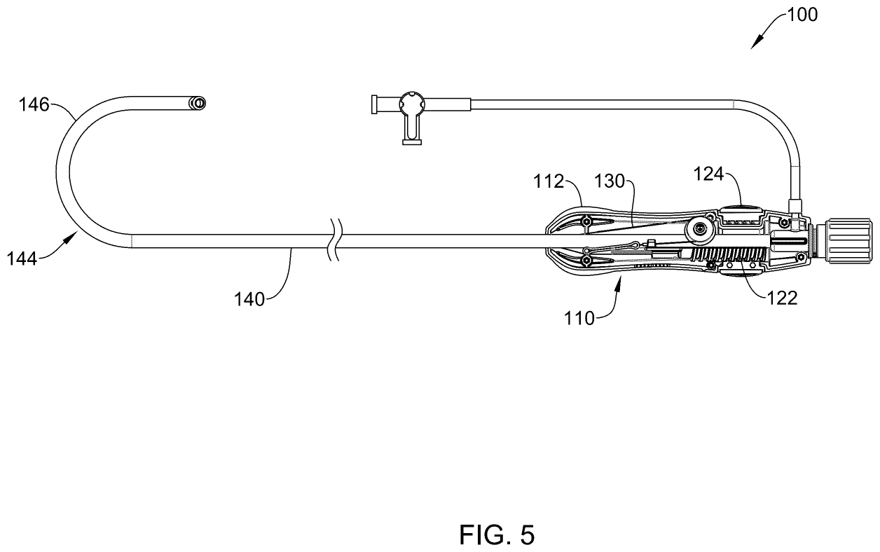

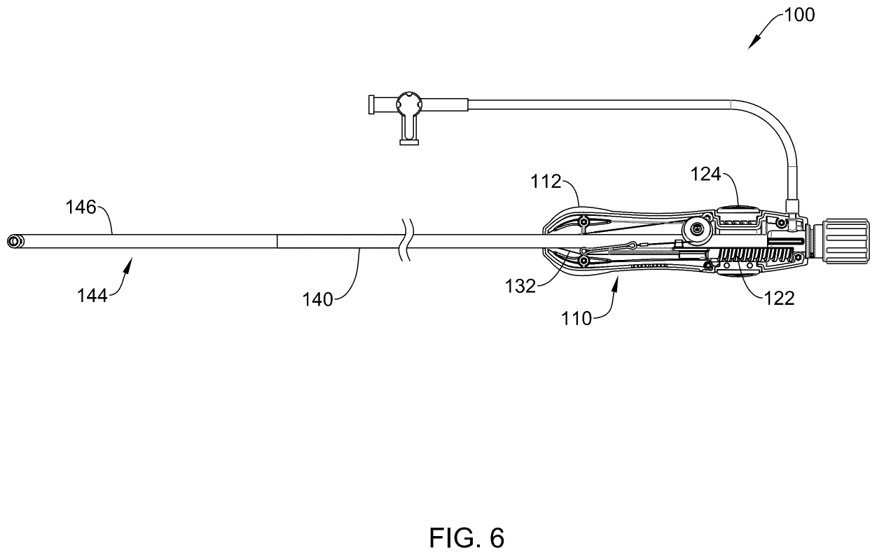

FIGS. 5 and 6 illustrate the relationship between certain features of the bi-directional steerable catheter 100 in the deflected and straightened configurations. As seen in FIG. 5, clockwise rotation of the rotatable knob 124, as viewed proximally to distally, has moved the threaded member 122 distally within the handle 110 and/or the handle housing 112, thereby applying tension to the first steering wire 130 and bending or deflecting the distal portion 144 and/or the first curve 146 of the elongate sheath 140 toward the handle 110 and/or the handle housing 112, or toward and/or to the deflected configuration. As seen in FIG. 6, counterclockwise rotation of the rotatable knob 124, as viewed proximally to distally, has moved the threaded member 122 proximally within the handle 110 and/or the handle housing 112, thereby applying tension to the second steering wire 132 and bending or deflecting the distal portion 144 and/or the first curve 146 of the elongate sheath 140 away from the handle 110 and/or the handle housing 112, or toward and/or to the straightened configuration. As discussed herein, other configurations are also contemplated.

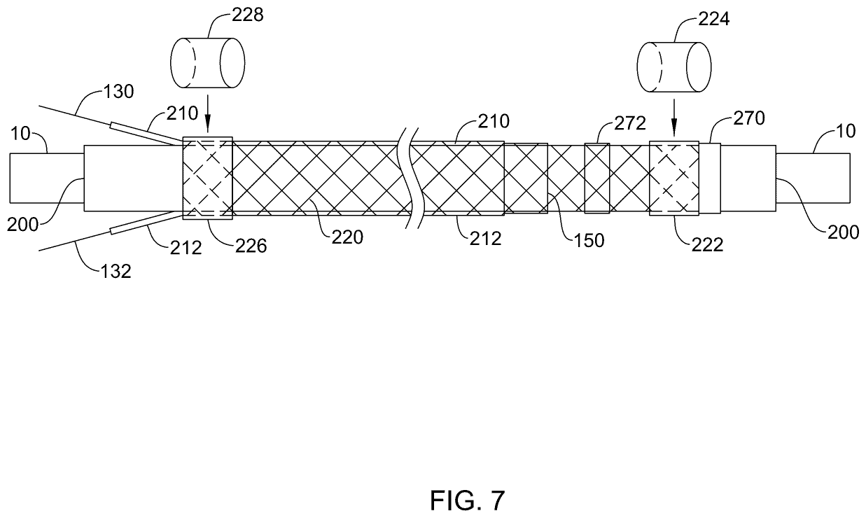

FIGS. 7-12 illustrate selected aspects and/or additional details related to the construction and/or manufacturing of the elongate sheath 140. In some embodiments, the elongate sheath 140 of and/or for the bi-directional steerable catheter 100 may comprise an inner tubular sleeve 200 having a proximal end and a distal end. As seen in FIG. 7, a method of making the elongate sheath 140 may comprise positioning the inner tubular sleeve 200 on a mandrel 10. After forming the elongate sheath 140, the mandrel 10 may be removed therefrom and/or the elongate sheath 140 may be removed from the mandrel 10 such that the inner tubular sleeve 200 defines the central lumen 143 and/or an inner surface of the wall 141. In some embodiments, the inner tubular sleeve 200 may be formed from a polymeric material. In some embodiments, the inner tubular sleeve 200 may be formed from two or more layers of polymeric material, wherein the two or more layers of polymeric material may each be different and/or discrete polymeric materials. In some embodiments, the two or more layers of polymeric material may be coextruded. In some embodiments, the two or more layers of polymeric material may be adhesively and/or molecularly bonded together. Other configurations are also contemplated. In one example, the inner tubular sleeve 200 may comprise a polytetrafluoroethylene (PTFE) liner or layer with a polyether block amide (e.g., Pebax®) layer disposed thereon and/or radially outward of the PTFE liner or layer. Other configurations and/or polymeric materials are also contemplated, and some non-limiting examples are described below.

In some embodiments, the elongate sheath 140 comprises the distal pull ring 150. As seen in FIG. 7, the distal pull ring 150 may be disposed radially outward of the inner tubular sleeve 200 and/or may be disposed on and/or over the inner tubular sleeve 200. The method of making the elongate sheath 140 may comprise positioning the distal pull ring 150 over the inner tubular sleeve 200 proximal of the distal end of the inner tubular sleeve 200. In some embodiments, the distal pull ring 150 may be at least temporarily attached to the inner tubular sleeve 200 such that the distal pull ring 150 is axially and/or longitudinally fixed in position along the inner tubular sleeve 200. In some embodiments, the distal pull ring 150 may be adhesively bonded to the inner tubular sleeve 200. In some embodiments, the distal pull ring 150 may be attached to the inner tubular sleeve 200 via reflow. Other configurations are also contemplated.

The first steering wire 130 and the second steering wire 132 may each extend proximally from the distal pull ring 150. In at least some embodiments, a distal end of the first steering wire 130 may be fixedly attached to the distal pull ring 150 and a distal end of the second steering wire 132 may be fixedly attached to the distal pull ring 150. The first steering wire 130 and the second steering wire 132 may be disposed radially outward of the inner tubular sleeve 200.

In some embodiments, the elongate sheath 140 may comprise a first steering wire tube 210 and a second steering wire tube 212 each extending proximally from the distal pull ring 150. The first steering wire 130 and the second steering wire 132 may be slidably disposed within the first steering wire tube 210 and the second steering wire tube 212, respectively. In at least some embodiments, the first steering wire tube 210 and the second steering wire tube 212 may define the plurality of steering wire lumens 145. The first steering wire tube 210 and the second steering wire tube 212 may be disposed radially outward of the inner tubular sleeve 200. The method of making the elongate sheath 140 may comprise positioning the first steering wire tube 210 and the second steering wire tube 212 over the inner tubular sleeve 200 and extending toward the proximal end of the inner tubular sleeve 200. In some embodiments, the first steering wire tube 210 and the second steering wire tube 212 may be at least temporarily attached to the inner tubular sleeve 200 such that the first steering wire tube 210 and the second steering wire tube 212 are circumferentially, axially, and/or longitudinally fixed in position along the inner tubular sleeve 200. In some embodiments, the first steering wire tube 210 and the second steering wire tube 212 may be adhesively bonded to the inner tubular sleeve 200. In some embodiments, the first steering wire tube 210 and the second steering wire tube 212 may be attached to the inner tubular sleeve 200 via reflow. Other configurations are also contemplated.

In some embodiments, the elongate sheath 140 may comprise a reinforcing braid 220 disposed over the inner tubular sleeve 200 between the proximal end of the inner tubular sleeve 200 and the distal end of the inner tubular sleeve 200, as seen in FIG. 7. In some embodiments, the method of making the elongate sheath 140 may comprise positioning the reinforcing braid 220 over the inner tubular sleeve 200, the distal pull ring 150, the first steering wire 130, and the second steering wire 132. As the first steering wire 130 may be disposed within the first steering wire tube 210 and the second steering wire 132 may be disposed within the second steering wire tube 212, the method of making the elongate sheath 140 may comprise positioning the reinforcing braid 220 over the first steering wire tube 210 and the second steering wire tube 212.

In some embodiments, the distal pull ring 150 may be disposed radially inward of the reinforcing braid 220. In some embodiments, the distal pull ring 150 may be disposed radially between the inner tubular sleeve 200 and the reinforcing braid 220. In some embodiments, the first steering wire 130 and the second steering wire 132 may be disposed radially inward of the reinforcing braid 220. In some embodiments, the first steering wire 130 and the second steering wire 132 may be disposed radially between the inner tubular sleeve 200 and the reinforcing braid 220. In some embodiments, the first steering wire tube 210 and the second steering wire tube 212 may be disposed radially inward of the reinforcing braid 220. In some embodiments, the first steering wire tube 210 and the second steering wire tube 212 may be disposed radially between the inner tubular sleeve 200 and the reinforcing braid 220.

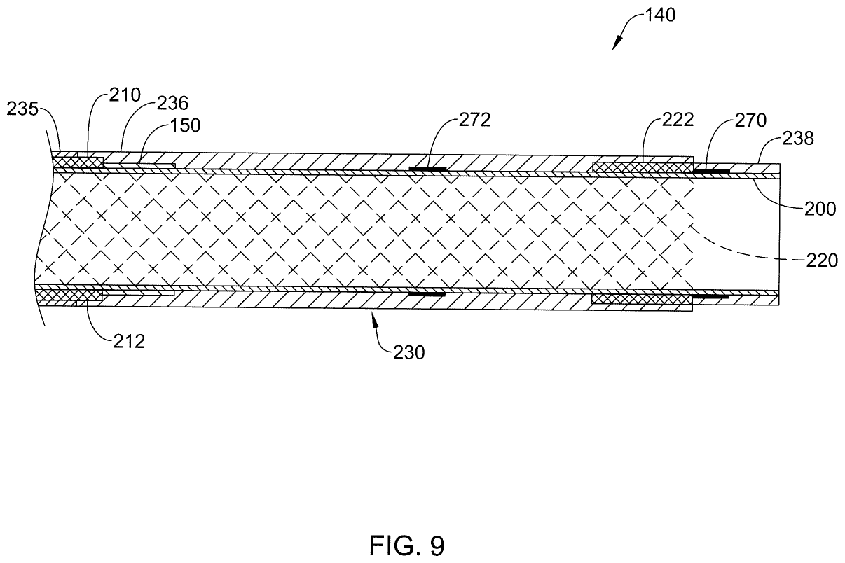

In some embodiments, the method of making the elongate sheath 140 and/or positioning the reinforcing braid 220 over the inner tubular sleeve 200 may comprise positioning a distal tubular braid tack 222 (e.g., FIGS. 7, 9) over the reinforcing braid 220 proximate, adjacent, and/or at the distal end of the reinforcing braid 220. In at least some embodiments, the distal tubular braid tack 222 may be positioned on and/or surrounding the distal end of the reinforcing braid 220. The distal tubular braid tack 222 may be formed from a polymeric material. In one example, the distal tubular braid tack 222 may be formed from polyether block amide (e.g., Pebax®). Other configurations and/or polymeric materials are also contemplated, and some non-limiting examples are described below. In some embodiments, the method of making the elongate sheath 140 and/or positioning the reinforcing braid 220 over the inner tubular sleeve 200 may comprise positioning a distal shrink wrap 224 over the distal tubular braid tack 222. In at least some embodiments, the method of making the elongate sheath 140 and/or positioning the reinforcing braid 220 over the inner tubular sleeve 200 may comprise applying heat to the distal shrink wrap 224 and the distal tubular braid tack 222 to reflow the distal tubular braid tack 222 around the reinforcing braid 220 proximate, adjacent, and/or at the distal end of the reinforcing braid 220. In some embodiments, the distal shrink wrap 224 may be subsequently removed.

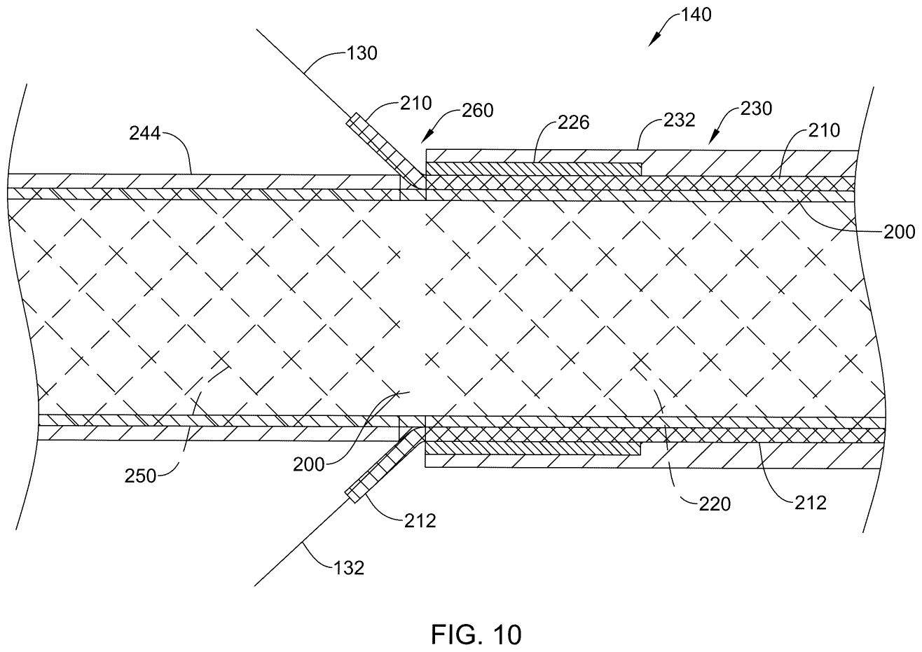

In some embodiments, the method of making the elongate sheath 140 and/or positioning the reinforcing braid 220 over the inner tubular sleeve 200 may comprise, after applying heat to the distal shrink wrap 224 and the distal tubular braid tack 222 to reflow the distal tubular braid tack 222 around the reinforcing braid 220, positioning a proximal tubular braid tack 226 (e.g., FIGS. 7, 10) over the reinforcing braid 220 proximate, adjacent, and/or at the proximal end of the reinforcing braid 220. In at least some embodiments, the proximal tubular braid tack 226 may be positioned on and/or surrounding the proximal end of the reinforcing braid 220. The proximal tubular braid tack 226 may be formed from a polymeric material. In one example, the proximal tubular braid tack 226 may be formed from polyether block amide (e.g., Pebax®). Other configurations and/or polymeric materials are also contemplated, and some non-limiting examples are described below. In some embodiments, the method of making the elongate sheath 140 and/or positioning the reinforcing braid 220 over the inner tubular sleeve 200 may comprise positioning a proximal shrink wrap 228 over the proximal tubular braid tack 226. In at least some embodiments, the method of making the elongate sheath 140 and/or positioning the reinforcing braid 220 over the inner tubular sleeve 200 may comprise applying heat to the proximal shrink wrap 228 and the proximal tubular braid tack 226 to reflow the proximal tubular braid tack 226 around the reinforcing braid 220 proximate, adjacent, and/or at the proximal end of the reinforcing braid 220. In some embodiments, the proximal shrink wrap 228 may be subsequently removed.

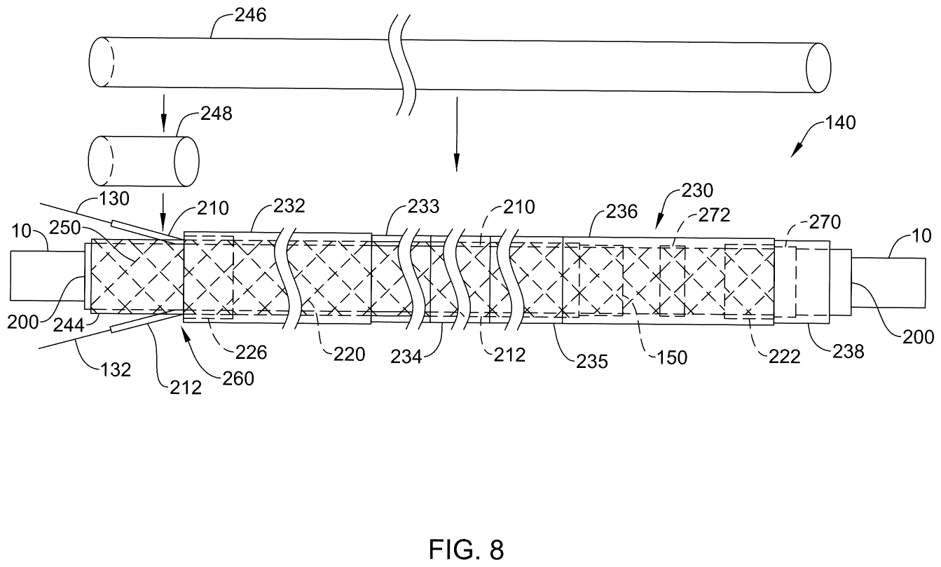

In some embodiments, the elongate sheath 140 may comprise an outer tubular sleeve 230 encompassing the reinforcing braid 220, as seen in FIG. 8. The outer tubular sleeve 230 may have a proximal end, a distal end, and a length defined by the proximal end and the distal end. In some embodiments, the method of making the elongate sheath 140 may comprise positioning the outer tubular sleeve 230 over the reinforcing braid 220. In at least some embodiments, the distal end of the reinforcing braid 220 may be spaced apart proximally from the distal end of the outer tubular sleeve 230. The outer tubular sleeve 230 may be formed from a polymeric material. In one example, the outer tubular sleeve 230 may be formed from polyether block amide (e.g., Pebax®). Other configurations and/or polymeric materials are also contemplated, and some non-limiting examples are described below.

In some embodiments, the outer tubular sleeve 230 may comprise a plurality of individual segments of polymeric material. In some embodiments, the plurality of individual segments of polymeric material may comprise varying physical characteristics and/or properties (e.g., hardness, stiffness, tensile strength, etc.). In some embodiments, positioning the outer tubular sleeve 230 over the reinforcing braid 220 may comprise positioning the plurality of individual segments of polymeric material over the reinforcing braid 220. In some embodiments, the plurality of individual segments of polymeric material may comprise at least three individual segments. In some embodiments, the plurality of individual segments of polymeric material may comprise at least four individual segments. In some embodiments, the plurality of individual segments of polymeric material may comprise at least five individual segments. In some embodiments, each individual segment of the plurality of individual segments may comprise the same polymeric material. In one example, each individual segment of the plurality of individual segments of polymeric material may comprise polyether block amide (e.g., Pebax®). In some alternative embodiments, one or more individual segments of the plurality of individual segments of polymeric material may comprise a different polymeric material than the remaining segments of the plurality of individual segments of polymeric material. Other configurations are also contemplated.

In some embodiments, at least three individual segments of the plurality of individual segments of polymeric material each have a different physical characteristic and/or property and alternate longitudinally along the length of the outer tubular sleeve 230 and/or the reinforcing braid 220 such that immediately adjacent individual segments have different values for a particular physical characteristic and/or property. In some embodiments, at least three individual segments of the plurality of individual segments of polymeric material each have a different hardness and alternate longitudinally along the length of the outer tubular sleeve 230 and/or the reinforcing braid 220 (e.g., first hardness, second hardness, first hardness (or a third hardness), second hardness, etc.). In some embodiments, at least three individual segments of the plurality of individual segments of polymeric material each have a different stiffness and alternate longitudinally along the length of the outer tubular sleeve 230 and/or the reinforcing braid 220. Other configurations are also contemplated.

In some embodiments, at least four individual segments of the plurality of individual segments of polymeric material each have a different physical characteristic and/or property and alternate longitudinally along the length of the outer tubular sleeve 230 and/or the reinforcing braid 220 such that immediately adjacent individual segments have different values for a particular physical characteristic and/or property. In some embodiments, at least four individual segments of the plurality of individual segments of polymeric material each have a different hardness and alternate longitudinally along the length of the outer tubular sleeve 230 and/or the reinforcing braid 220 (e.g., first hardness, second hardness, first hardness (or a third hardness), second hardness, etc.). In some embodiments, at least four individual segments of the plurality of individual segments of polymeric material each have a different stiffness and alternate longitudinally along the length of the outer tubular sleeve 230 and/or the reinforcing braid 220. Other configurations are also contemplated.

In some embodiments, at least five individual segments of the plurality of individual segments of polymeric material each have a different physical characteristic and/or property and alternate longitudinally along the length of the outer tubular sleeve 230 and/or the reinforcing braid 220 such that immediately adjacent individual segments have different values for a particular physical characteristic and/or property. In some embodiments, at least five individual segments of the plurality of individual segments of polymeric material each have a different hardness and alternate longitudinally along the length of the outer tubular sleeve 230 and/or the reinforcing braid 220 (e.g., first hardness, second hardness, first hardness (or third hardness), second hardness, etc.). In some embodiments, at least five individual segments of the plurality of individual segments of polymeric material each have a different stiffness and alternate longitudinally along the length of the outer tubular sleeve 230 and/or the reinforcing braid 220. Other configurations are also contemplated.

In one non-limiting example, the plurality of individual segments of polymeric material may comprise a first segment 232 having a Shore hardness of 72D, a second segment 233 having a Shore hardness of 45D, a third segment 234 having a Shore hardness of 63D, a fourth segment 235 having a Shore hardness of 40D, a fifth segment 236 having a Shore hardness of 63D, and a sixth segment 238 having a Shore hardness of 35D. Other configurations, values, and/or properties are also contemplated.

In some embodiments, the outer tubular sleeve 230 may comprise a proximal handle segment 244 of polymeric material disposed over the inner tubular sleeve 200 proximal of the reinforcing braid 220. In some embodiments, the proximal handle segment 244 of polymeric material may have a Shore hardness of 72D. Other configurations, values, and/or properties are also contemplated. In some embodiments, two proximalmost adjacent segments of the plurality of individual segments of polymeric material may have a similar Shore hardness or the same Shore hardness. In some embodiments, two distalmost adjacent segments of the plurality of individual segments of polymeric material may have a similar Shore hardness or the same Shore hardness. Other configurations are also contemplated.

In some embodiments, positioning the outer tubular sleeve 230 over the reinforcing braid 220 may comprise positioning the proximal handle segment 244 of polymeric material over inner tubular sleeve 200 proximal of the reinforcing braid 220. In some embodiments, the proximal handle segment 244 of polymeric material may comprise a second reinforcing braid 250 embedded therein. In some embodiments, the second reinforcing braid 250 may be embedded within the proximal handle segment 244 of polymeric material prior to use and/or prior to positioning the proximal handle segment 244 of polymeric material over inner tubular sleeve 200 proximal of the reinforcing braid 220. In some embodiments, positioning the outer tubular sleeve 230 over the reinforcing braid 220 may comprise positioning the second reinforcing braid 250 over the inner tubular sleeve 200 proximal of the reinforcing braid 220, and thereafter, positioning the proximal handle segment 244 of polymeric material over the second reinforcing braid 250 proximal of the reinforcing braid 220. Other configurations are also contemplated.

In some embodiments, the first steering wire 130 and the second steering wire 132 may each be disposed and/or may extend between the inner tubular sleeve 200 and the reinforcing braid 220 from the distal pull ring 150 proximally to an exit location 260 (e.g., FIGS. 8, 10) disposed distal of the proximal end of the inner tubular sleeve 200 and proximal of a proximal end of the reinforcing braid 220. In some embodiments, the first steering wire tube 210 and the second steering wire tube 212 may each be disposed and/or may extend between the inner tubular sleeve 200 and the reinforcing braid 220 from the distal pull ring 150 proximally to the exit location 260 disposed distal of the proximal end of the inner tubular sleeve 200 and proximal of the proximal end of the reinforcing braid 220. The proximal end of the reinforcing braid 220 may be disposed distal of the exit location 260. The proximal end of the outer tubular sleeve 230 may be disposed proximal of the exit location 260, and/or the exit location 260 may be disposed distal of the proximal end of the outer tubular sleeve 230. In some embodiments, the proximal handle segment 244 of polymeric material may be disposed proximal of the exit location 260. In some embodiments, the exit location 260 may be disposed distal of the second reinforcing braid 250. In at least some embodiments, the exit location 260 may be disposed axially and/or longitudinally between the reinforcing braid 220 and the second reinforcing braid 250. In some embodiments, the reinforcing braid 220 and the second reinforcing braid 250 may be axially and/or longitudinally spaced apart from one another at and/or adjacent the exit location, as seen in FIG. 10.

In some embodiments, the exit location 260 may be defined as a location where the first steering wire 130 and the second steering wire 132 shift from radially inward of the reinforcing braid 220 and/or the outer tubular sleeve 230 to radially outward of the outer tubular sleeve 230. In some embodiments, the exit location 260 may be defined as a location where the first steering wire 130 and the second steering wire 132 extend radially through the outer tubular sleeve 230. In some embodiments, the exit location 260 may be defined as a location where the first steering wire tube 210 and the second steering wire tube 212 shift from radially inward of the reinforcing braid 220 and/or the outer tubular sleeve 230 to radially outward of the outer tubular sleeve 230. In some embodiments, the exit location 260 may be defined as a location where the first steering wire tube 210 and the second steering wire tube 212 extend radially through the outer tubular sleeve 230. The first steering wire 130 and the second steering wire 132 may extend proximally of and/or from the exit location. The first steering wire 130 and the second steering wire 132 may extend proximally of and/or from the exit location outside of and/or radially outward of the outer tubular sleeve 230, the proximal handle segment 244, and/or the second reinforcing braid 250. The first steering wire tube 210 and the second steering wire tube 212 may extend proximally of and/or from the exit location. The first steering wire tube 210 and the second steering wire tube 212 may extend proximally of and/or from the exit location outside of and/or radially outward of the outer tubular sleeve 230, the proximal handle segment 244, and/or the second reinforcing braid 250.

In at least some embodiments, the first steering wire tube 210 and the second steering wire tube 212 may each terminate proximal of the proximal end of the reinforcing braid 220, distal of the proximal end of the inner tubular sleeve 200, and/or distal of the proximal end of the outer tubular sleeve 230. In some embodiments, a proximal end of the first steering wire tube 210 and a proximal end of the second steering wire tube 212 may be disposed proximal of the proximal end of the reinforcing braid 220, distal of the proximal end of the inner tubular sleeve 200, and/or distal of the proximal end of the outer tubular sleeve 230. The first steering wire 130 and the second steering wire 132 may extend proximally from the proximal end of the first steering wire tube 210 and the proximal end of the second steering wire tube 212, respectively.

In some embodiments, the elongate sheath 140 may comprise a distal marker band 270 disposed distal of the distal end of the reinforcing braid 220, proximal of the distal end of the inner tubular sleeve 200, and proximal of the distal end of the outer tubular sleeve 230, as seen in at least FIG. 9. In at least some embodiments, the distal marker band 270 may be disposed radially outward of the inner tubular sleeve 200 and radially inward of the outer tubular sleeve 230. In some embodiments, the method of making the elongate sheath 140 may comprise positioning the distal marker band 270 over and/or on the inner tubular sleeve 200 proximal of the distal end of the inner tubular sleeve 200. In some embodiments, the method of making the elongate sheath 140 may comprise positioning the distal marker band 270 over and/or on the inner tubular sleeve 200 proximal of the distal end of the inner tubular sleeve 200 prior to positioning the reinforcing braid 220 over the inner tubular sleeve 200, the distal pull ring 150, the first steering wire 130, and the second steering wire 132.

In some embodiments, the distal marker band 270 may be at least temporarily attached to the inner tubular sleeve 200 such that the distal marker band 270 is axially and/or longitudinally fixed in position along the inner tubular sleeve 200. In some embodiments, the distal marker band 270 may be adhesively bonded to the inner tubular sleeve 200. In some embodiments, the distal marker band 270 may be attached to the inner tubular sleeve 200 via reflow. Other configurations are also contemplated.

In some embodiments, the method of making the elongate sheath 140 may comprise positioning a distal marker band shrink wrap over the distal marker band 270, and thereafter, applying heat to the distal marker band shrink wrap, the distal marker band 270, and the inner tubular sleeve 200 to secure the distal marker band 270 to the inner tubular sleeve 200.

In some embodiments, the elongate sheath 140 may comprise a proximal marker band 272 disposed proximal of the distal end of the reinforcing braid 220, proximal of the distal end of the inner tubular sleeve 200, and proximal of the distal end of the outer tubular sleeve 230. In at least some embodiments, the proximal marker band 272 may be disposed radially outward of the inner tubular sleeve 200, radially inward of the reinforcing braid 220, and radially inward of the outer tubular sleeve 230. In some embodiments, the proximal marker band 272 may be spaced apart proximally from the distal marker band 270 and spaced apart distally from the distal pull ring 150.

In some embodiments, the method of making the elongate sheath 140 may comprise positioning the proximal marker band 272 over and/or on the inner tubular sleeve 200 proximal of the distal end of the inner tubular sleeve 200. In some embodiments, the method of making the elongate sheath 140 may comprise positioning the reinforcing braid 220 over the proximal marker band 272. In some embodiments, the method of making the elongate sheath 140 may comprise positioning the proximal marker band 272 over and/or on the inner tubular sleeve 200 proximal of the distal end of the inner tubular sleeve 200 prior to positioning the reinforcing braid 220 over the inner tubular sleeve 200, the distal pull ring 150, the first steering wire 130, the second steering wire 132, and the proximal marker band 272.

In some embodiments, the proximal marker band 272 may be at least temporarily attached to the inner tubular sleeve 200 such that the proximal marker band 272 is axially and/or longitudinally fixed in position along the inner tubular sleeve 200. In some embodiments, the proximal marker band 272 may be adhesively bonded to the inner tubular sleeve 200. In some embodiments, the proximal marker band 272 may be attached to the inner tubular sleeve 200 via reflow. Other configurations are also contemplated.

In some embodiments, the method of making the elongate sheath 140 may comprise positioning a proximal marker band shrink wrap over the proximal marker band 272, and thereafter, applying heat to the proximal marker band shrink wrap, the proximal marker band 272, and the inner tubular sleeve 200 to secure the proximal marker band 272 to the inner tubular sleeve 200.

In some embodiments, the method of making the elongate sheath 140 may comprise positioning the proximal marker band shrink wrap over the proximal marker band 272 and positioning the distal marker band shrink wrap over the distal marker band 270 before applying heat to the distal marker band shrink wrap, the distal marker band 270, the proximal marker band shrink wrap, the proximal marker band 272, and the inner tubular sleeve 200 to secure the distal marker band 270 and the proximal marker band 272 to the inner tubular sleeve 200 in a single step, operation, or process. In at least some embodiments, the distal marker band shrink wrap and the proximal marker band shrink wrap may be separate and distinct structures. In some embodiments, the distal marker band shrink wrap and the proximal marker band shrink wrap may be axially and/or longitudinally spaced apart from each other.

In some embodiments, the method of making the elongate sheath 140 may comprise removing the proximal marker band shrink wrap and removing the distal marker band shrink wrap after applying heat to secure the distal marker band 270 and the proximal marker band 272 to the inner tubular sleeve 200. In some embodiments, the method of making the elongate sheath 140 and/or positioning the outer tubular sleeve 230 over the reinforcing braid 220 may comprise positioning the outer tubular sleeve 230 over the distal marker band 270 and the proximal marker band 272. In some embodiments, the method of making the elongate sheath 140 and/or positioning the outer tubular sleeve 230 over the reinforcing braid 220 may comprise positioning the outer tubular sleeve 230 over the distal marker band 270 and the proximal marker band 272 after removing the proximal marker band shrink wrap and the distal marker band shrink wrap.

In some embodiments, the method of making the elongate sheath 140 may comprise, after positioning the outer tubular sleeve 230 over the reinforcing braid 220, positioning a shrink wrap 246 over the outer tubular sleeve 230, and/or surrounding the outer tubular sleeve 230 with the shrink wrap 246, as seen in FIG. 8. In some embodiments, the method of making the elongate sheath 140 may comprise, after positioning the outer tubular sleeve 230 over the reinforcing braid 220 and/or after positioning the proximal handle segment 244 of polymeric material over inner tubular sleeve 200 proximal of the reinforcing braid 220, positioning a proximal handle shrink wrap 248 over the proximal handle segment 244 of polymeric material, as seen in FIG. 8. In some embodiments, the method of making the elongate sheath 140 may comprise positioning the shrink wrap 246 over the outer tubular sleeve 230 and the proximal handle shrink wrap 248, and/or surrounding the outer tubular sleeve 230 and the proximal handle shrink wrap 248 with the shrink wrap 246.

The method of making the elongate sheath 140 may comprise applying heat to the shrink wrap 246 over the outer tubular sleeve 230, and/or surrounding the outer tubular sleeve 230, to reflow the outer tubular sleeve 230 around the reinforcing braid 220 and the inner tubular sleeve 200. In some embodiments, applying heat to the shrink wrap 246 over the outer tubular sleeve 230, and/or surrounding the outer tubular sleeve 230, may reflow the proximal handle segment 244 of polymeric material with the plurality of individual segments of polymeric material. In some embodiments, applying heat to the shrink wrap 246 over the outer tubular sleeve 230, and/or surrounding the outer tubular sleeve 230, may reflow the proximal handle segment 244 of polymeric material with the first segment 232. In some embodiments, applying heat to the shrink wrap 246 over the outer tubular sleeve 230, and/or surrounding the outer tubular sleeve 230, may reflow the proximal handle segment 244 of polymeric material with the first segment 232 at and/or adjacent the exit location 260.

In some embodiments, the method of making the elongate sheath 140 may comprise, thereafter, removing the shrink wrap 246 over the outer tubular sleeve 230, and/or surrounding the outer tubular sleeve 230. In some embodiments, the method of making the elongate sheath 140 may comprise, thereafter, removing the shrink wrap 246 over the outer tubular sleeve 230, and/or surrounding the outer tubular sleeve 230, and removing the proximal handle shrink wrap 248.

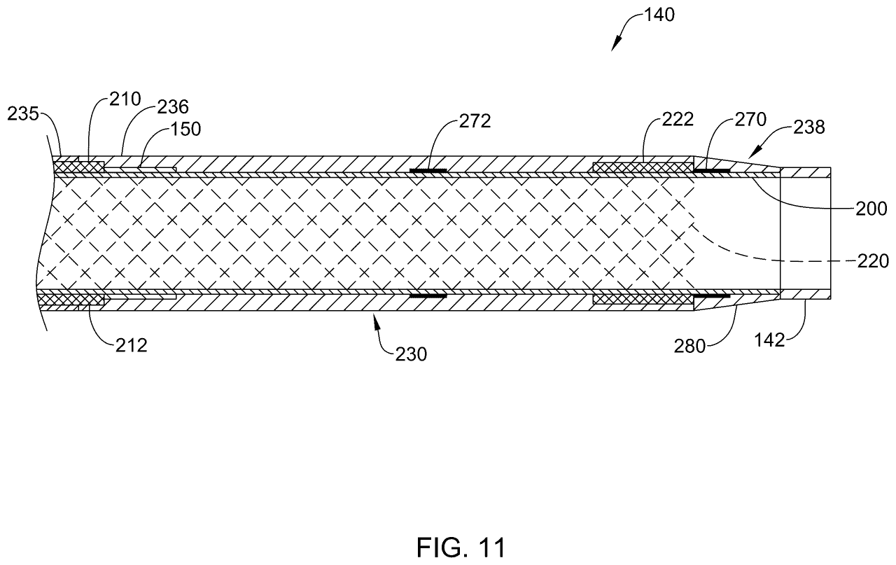

In some embodiments, applying heat to the shrink wrap 246 over the outer tubular sleeve 230, and/or surrounding the outer tubular sleeve 230, to reflow the outer tubular sleeve 230 around the reinforcing braid 220 and the inner tubular sleeve 200 may cause and/or form a tapered distal region 280, as seen in FIG. 11. In some embodiments, the tapered distal region 280 may be disposed proximate and/or adjacent to the distal end of the inner tubular sleeve 200 and/or the distal end of the outer tubular sleeve 230. In some embodiments, the tapered distal region 280 may be disposed proximate and/or adjacent to the distal end of the reinforcing braid 220. In some embodiments, the tapered distal region 280 may be disposed distal of the distal end of the reinforcing braid 220. In some embodiments, the sixth segment 238 may be and/or include the tapered distal region 280.

In some embodiments, the elongate sheath 140 may comprise the soft and/or atraumatic distal tip 142 disposed distal of the tapered distal region 280. In some embodiments, the soft and/or atraumatic distal tip 142 may be formed from a polymeric material. In some embodiments, the soft and/or atraumatic distal tip 142 may comprise polyether block amide (e.g., Pebax®). In some embodiments, the soft and/or atraumatic distal tip 142 may have a Shore hardness of 35D. Other configurations, values, and/or properties are also contemplated.

In some embodiments, the method of making the elongate sheath 140 may comprise securing and/or fixedly attaching the soft and/or atraumatic distal tip 142 to and/or at the distal end of the inner tubular sleeve 200 and/or the distal end of the outer tubular sleeve 230. In some embodiments, the method of making the elongate sheath 140 may comprise securing and/or fixedly attaching the soft and/or atraumatic distal tip 142 to and/or at the distal end of the inner tubular sleeve 200 and/or the distal end of the outer tubular sleeve 230 via reflow. In some embodiments, the method of making the elongate sheath 140 may comprise securing and/or fixedly attaching the soft and/or atraumatic distal tip 142 to and/or at the distal end of the inner tubular sleeve 200 and/or the distal end of the outer tubular sleeve 230 via adhesive bonding. Other configurations are also contemplated.

In some embodiments, the method of making the elongate sheath 140 may comprise securing and/or fixedly attaching the soft and/or atraumatic distal tip 142 to and/or at the distal end of the inner tubular sleeve 200 and/or the distal end of the outer tubular sleeve 230 after applying heat to the shrink wrap 246 over the outer tubular sleeve 230, and/or surrounding the outer tubular sleeve 230, to reflow the outer tubular sleeve 230 around the reinforcing braid 220 and the inner tubular sleeve 200. In some embodiments, the method of making the elongate sheath 140 may comprise securing and/or fixedly attaching the soft and/or atraumatic distal tip 142 to and/or at the distal end of the inner tubular sleeve 200 and/or the distal end of the outer tubular sleeve 230 before applying heat to the shrink wrap 246 over the outer tubular sleeve 230, and/or surrounding the outer tubular sleeve 230, to reflow the outer tubular sleeve 230 around the reinforcing braid 220 and the inner tubular sleeve 200. In some embodiments, the method of making the elongate sheath 140 may comprise securing and/or fixedly attaching the soft and/or atraumatic distal tip 142 to and/or at the distal end of the inner tubular sleeve 200 and/or the distal end of the outer tubular sleeve 230 before positioning the shrink wrap 246 over the outer tubular sleeve 230, and/or before surrounding the outer tubular sleeve 230 with the shrink wrap 246.

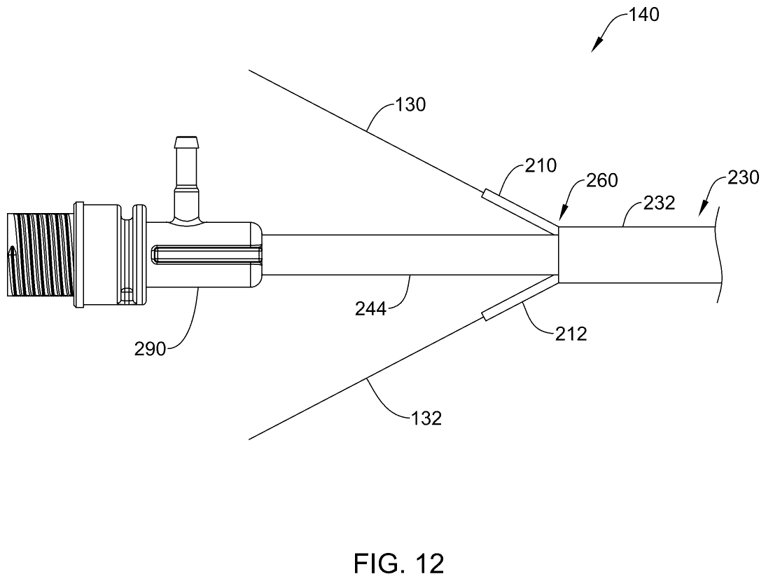

In some embodiments, the elongate sheath 140 may comprise a hub member 290 fixedly attached to a proximal end of the outer tubular sleeve 230, as seen in FIG. 12. In some embodiments, the elongate sheath 140 may comprise a hub member 290 fixedly attached to a proximal end of the proximal handle segment 244. In some embodiments, the method of making the elongate sheath 140 may comprise fixedly attaching the hub member 290 to the proximal end of the outer tubular sleeve 230. In some embodiments, the method of making the elongate sheath 140 may comprise fixedly attaching the hub member 290 to the proximal end of the proximal handle segment 244. In some embodiments, the hub member 290 may be fixedly attached to the outer tubular sleeve 230 and/or the proximal handle segment 244 via reflow. In some embodiments, the hub member 290 may be fixedly attached to the outer tubular sleeve 230 and/or the proximal handle segment 244 via adhesive bonding. In some embodiments, the hub member 290 may be fixedly attached to the outer tubular sleeve 230 and/or the proximal handle segment 244 via welding. Other configurations are also contemplated. In some alternative embodiments, the hub member 290 may be integrally and/or monolithically formed with the outer tubular sleeve 230 and/or the proximal handle segment 244. In some embodiments, the outer tubular sleeve 230 and/or the proximal handle segment 244 may be integrally and/or monolithically formed with the hub member 290 disposed at the proximal end thereof.

In some embodiments, the elongate sheath 140 and/or components thereof may be heat treated to form and/or define the normal configuration or the relaxed configuration. As discussed herein, the elongate sheath 140 may include a preset double curve in the normal configuration or the relaxed configuration, as seen in FIGS. 1 and 4. In some embodiments, the method of making the elongate sheath 140 may comprise heat treating the elongate sheath 140 and/or the components thereof to form and/or define the normal configuration or the relaxed configuration. In some embodiments, the method of making the elongate sheath 140 may comprise heat treating the elongate sheath 140 and/or the components thereof before fixedly attaching the hub member 290 to the outer tubular sleeve 230 and/or the proximal handle segment 244. In some embodiments, the method of making the elongate sheath 140 may comprise heat treating the elongate sheath 140 and/or the components thereof after fixedly attaching the hub member 290 to the outer tubular sleeve 230 and/or the proximal handle segment 244. In some embodiments, in the normal configuration or the relaxed configuration of the elongate sheath 140, the preset double curve may be disposed between the proximal end of the reinforcing braid 220 and the distal end of the reinforcing braid 220. In some embodiments, outer tubular sleeve 230 and/or the reinforcing braid 220 may at least partially define the normal configuration or the relaxed configuration after heat treating. For example, in some embodiments, the outer tubular sleeve 230 and/or the reinforcing braid 220 may be formed from a shape memory material. Other configurations and/or materials are also contemplated.

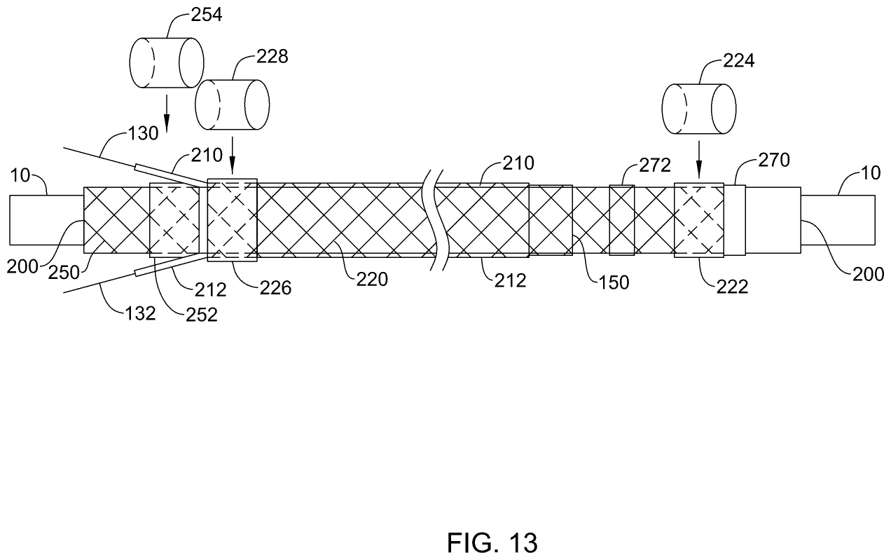

FIG. 13 illustrates selected aspects of an alternative configuration of the elongate sheath 140 and/or alternative aspects of the method of making the elongate sheath 140. As discussed herein, in some embodiments, positioning the outer tubular sleeve 230 (not shown in FIG. 13) over the reinforcing braid 220 may comprise positioning the second reinforcing braid 250 over the inner tubular sleeve 200 proximal of the reinforcing braid 220, and thereafter, positioning the proximal handle segment 244 of polymeric material over the second reinforcing braid 250 proximal of the reinforcing braid 220. In some embodiments, positioning the second reinforcing braid 250 over the inner tubular sleeve 200 may comprise positioning a tubular braid tack 252 over the second reinforcing braid 250 proximate, adjacent, and/or at the distal end of the second reinforcing braid 250, as shown in FIG. 13. In at least some embodiments, the tubular braid tack 252 may be positioned on and/or surrounding the distal end of the second reinforcing braid 250. The tubular braid tack 252 may be formed from a polymeric material. In one example, the tubular braid tack 252 may be formed from polyether block amide (e.g., Pebax®). Other configurations and/or polymeric materials are also contemplated, and some non-limiting examples are described below. In some embodiments, the method of making the elongate sheath 140 and/or positioning the second reinforcing braid 250 over the inner tubular sleeve 200 may comprise positioning a second reinforcing braid shrink wrap 254 over the tubular braid tack 252. In at least some embodiments, the method of making the elongate sheath 140 and/or positioning the second reinforcing braid 250 over the inner tubular sleeve 200 may comprise applying heat to the second reinforcing braid shrink wrap 254 and the tubular braid tack 252 to reflow the tubular braid tack 252 around the second reinforcing braid 250 proximate, adjacent, and/or at the distal end of the second reinforcing braid 250. In some embodiments, the second reinforcing braid shrink wrap 254 may be subsequently removed. Thereafter, the proximal handle segment 244 and/or the outer tubular sleeve 230 may be positioned over the reinforcing braid 220 and the second reinforcing braid 250.

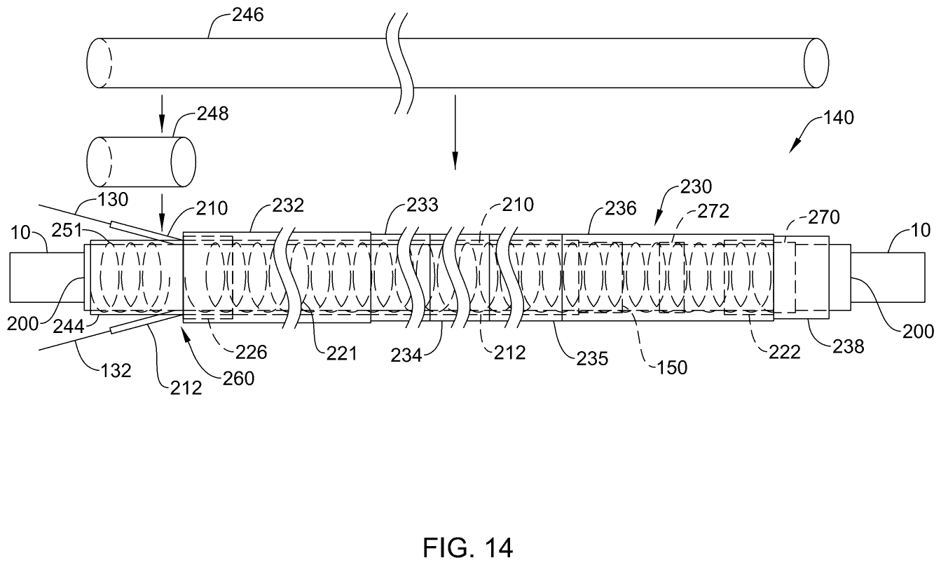

FIG. 14 illustrates selected aspects of another alternative configuration of the elongate sheath 140. In some embodiments, the reinforcing braid 220 (e.g., FIGS. 7-11, 13) may be and/or may be replaced with a reinforcing coil 221. In some embodiments, the second reinforcing braid 250 (e.g., FIGS. 8, 10, 13) may be and/or may be replaced with a second reinforcing coil 251. Construction of the elongate sheath 140 and/or the method of making the elongate sheath 140 may be substantially as described herein while substituting the reinforcing coil 221 for the reinforcing braid 220 and substituting the second reinforcing coil 251 for the second reinforcing braid 250.

The materials that can be used for the various components of the system (and/or other elements disclosed herein) and the various components thereof disclosed herein may include those commonly associated with medical devices and/or systems. For simplicity purposes, the following discussion refers to the system. However, this is not intended to limit the devices and methods described herein, as the discussion may be applied to other elements, members, components, or devices disclosed herein, such as, but not limited to, the occlusive implant, the delivery sheath, the core wire, the expandable framework, the occlusive element, the capsule, the elongate fingers, the elongate strand, etc. and/or elements or components thereof.

In some embodiments, the system and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly (alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate ((PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, the system and/or components thereof may include a fabric material. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or components thereof may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or components thereof may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine);

antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An elongate sheath for a bi-directional steerable catheter, comprising:
an inner tubular sleeve having a proximal end and a distal end;
a reinforcing braid disposed over the inner tubular sleeve between the proximal end of the inner tubular sleeve and the distal end of the inner tubular sleeve;
a distal pull ring disposed radially between the inner tubular sleeve and the reinforcing braid;
a first steering wire and a second steering wire each extending proximally from the distal pull ring; and
an outer tubular sleeve encompassing the reinforcing braid, the outer tubular sleeve having a proximal end, a distal end, and a length defined by the proximal end and the distal end;
wherein the first steering wire and the second steering wire extend between the inner tubular sleeve and the reinforcing braid from the distal pull ring proximally to an exit location disposed distal of the proximal end of the inner tubular sleeve;
wherein the proximal end of the outer tubular sleeve is disposed proximal of the exit location.

2. The elongate sheath of claim 1, wherein a distal end of the reinforcing braid is spaced apart proximally from the distal end of the outer tubular sleeve and a proximal end of the reinforcing braid is disposed distal of the exit location.

3. The elongate sheath of claim 1, wherein the first steering wire and the second steering wire are fixedly attached to the distal pull ring.

4. The elongate sheath of claim 1, further comprising a first steering wire tube and a second steering wire tube each extending proximally from the distal pull ring to the exit location, the first steering wire and the second steering wire being slidably disposed within the first steering wire tube and the second steering wire tube, respectively.

5. The elongate sheath of claim 1, further comprising a distal marker band disposed distal of the distal end of the reinforcing braid and proximal of the distal end of the outer tubular sleeve.

6. The elongate sheath of claim 5, further comprising a proximal marker band spaced apart proximally from the distal marker band and spaced apart distally from the distal pull ring.

7. An elongate sheath for a bi-directional steerable catheter, comprising:
an inner tubular sleeve having a proximal end and a distal end;
a reinforcing braid disposed over the inner tubular sleeve between the proximal end of the inner tubular sleeve and the distal end of the inner tubular sleeve;
a distal pull ring disposed radially between the inner tubular sleeve and the reinforcing braid;
a first steering wire and a second steering wire each extending proximally from the distal pull ring;
an outer tubular sleeve encompassing the reinforcing braid, the outer tubular sleeve having a proximal end, a distal end, and a length defined by the proximal end and the distal end;
wherein the first steering wire and the second steering wire extend between the inner tubular sleeve and the reinforcing braid from the distal pull ring proximally to an exit location disposed distal of the proximal end of the inner tubular sleeve;
wherein the proximal end of the outer tubular sleeve is disposed proximal of the exit location; and
a second reinforcing braid spaced apart from the reinforcing braid, wherein the second reinforcing braid is disposed over the inner tubular sleeve proximal of the exit location and encompassed by the outer tubular sleeve.

8. The elongate sheath of claim 7, wherein the first steering wire and the second steering wire are disposed radially outward of the outer tubular sleeve proximal of the exit location.

9. The elongate sheath of claim 7, wherein in a normal configuration of the elongate sheath, a preset double curve is disposed between a proximal end of the reinforcing braid and a distal end of the reinforcing braid.

10. The elongate sheath of claim 7, wherein the outer tubular sleeve is formed from a plurality of individual segments of polymeric material;
wherein at least three individual segments of the plurality of individual segments each have a different hardness and alternate longitudinally along the length of the outer tubular sleeve.

11. A method of making an elongate sheath for a bi-directional steerable catheter, comprising:
positioning an inner tubular sleeve on a mandrel;
positioning a distal pull ring over the inner tubular sleeve proximal of a distal end of the inner tubular sleeve, wherein a first steering wire and a second steering wire each extend proximally from the distal pull ring;
positioning a reinforcing braid over the inner tubular sleeve, the distal pull ring, the first steering wire, and the second steering wire;
positioning an outer tubular sleeve over the reinforcing braid;
surrounding the outer tubular sleeve with a shrink wrap; and
applying heat to reflow the outer tubular sleeve around the reinforcing braid and the inner tubular sleeve.

12. The method of claim 11, wherein positioning the reinforcing braid further comprises:
positioning a distal tubular braid tack over the reinforcing braid proximate a distal end of the reinforcing braid;
positioning a distal shrink wrap over the distal tubular braid tack; and
applying heat to reflow the distal tubular braid tack around the reinforcing braid proximate the distal end of the reinforcing braid.

13. The method of claim 12, wherein positioning the reinforcing braid further comprises, after applying heat to reflow the distal tubular braid tack:
positioning a proximal tubular braid tack over the reinforcing braid proximate a proximal end of the reinforcing braid;
positioning a proximal shrink wrap over the proximal tubular braid tack; and
applying heat to reflow the proximal tubular braid tack around the reinforcing braid proximate the proximal end of the reinforcing braid.

14. The method of claim 11, wherein positioning the outer tubular sleeve further comprises:
positioning a plurality of individual segments of polymeric material over the reinforcing braid;
wherein at least three individual segments of the plurality of individual segments each have a different hardness and alternate longitudinally along the length of the outer tubular sleeve.

15. The method of claim 14, wherein positioning the outer tubular sleeve further comprises positioning a proximal handle segment of polymeric material over the inner tubular sleeve proximal of the reinforcing braid.

16. The method of claim 15, wherein positioning the outer tubular sleeve further comprises positioning a second reinforcing braid over the inner tubular sleeve proximal of the reinforcing braid; and
thereafter, positioning the proximal handle segment of polymeric material over the second reinforcing braid.

17. The method of claim 15, wherein the proximal handle segment of polymeric material comprises a second reinforcing braid embedded therein.

18. The method of claim 15, further comprising fixedly attaching a hub member to a proximal end of the outer tubular sleeve.

19. The method of claim 15, wherein the proximal handle segment is monolithically formed with a hub member disposed at a proximal end thereof.

20. The method of claim 15, wherein a proximal portion of the first steering wire and a proximal portion of the second steering wire are disposed radially outward of the proximal handle segment of polymeric material.
